(19) United States
(12) Patent Application Publication
(10) Pub. No.: US 2012/0199118 A1
(43) Pub. Date: Aug. 9, 2012

(54) BOTTLE CLOSURE WITH HINGED SEAL

(75) Inventor: Michael Cuzydlo, Orchard Park, NY (US)

(73) Assignee: Piramal Critical Care, Inc., Orchard Park, NY (US)

(21) Appl. No.: 13/364,923

(22) Filed: Feb. 2, 2012

Related U.S. Application Data
(60) Provisional application No. 61/439,523, filed on Feb. 4, 2011.

(51) Int. Cl. A61M 16/01

(52) U.S. Cl. 128/200.14

(57) ABSTRACT

An anesthetic container and closure for transferring anesthesia from a container to a vaporizer. A hinged closure member is moveable between an open position and a closed position by a receiving member of a vaporizer when the anesthetic container is attached to the vaporizer for transfer of liquid anesthetic from the anesthetic container to the vaporizer. The hinged closure member is moved to the closed position upon withdrawal of the receiving member.

### RELATED APPLICATION DATA

This application claims benefit from U.S. Provisional Application 61/439,523, filed on Feb. 4, 2011, the disclosure of which is incorporated herein by reference in its entirety.

### BACKGROUND OF THE INVENTION

Liquid anesthetics are often packaged in glass bottles and shipped to a location where they may be used to anesthetize a patient undergoing a medical or dental procedure. Such anesthetics may also be used to induce analgesia in a patient undergoing a medical or dental procedure. In order to administer the anesthetic, the contents of the glass bottle are placed in a vaporizer. The vaporizer can vaporize the anesthetic and provide the vaporized anesthetic in a desired amount to the patient.

Inhalable anesthetics are typically volatile substances with relatively low boiling points and high vapor pressure. Preferably, there is little or no release of anesthetic to the atmosphere during handing. To transfer liquid anesthetic to a vaporizer, however, the bottle containing the vaporizer must be opened. Since it is unwise to expose medical personnel performing a procedure to an anesthetic, and since anesthetics are expensive, devices have been developed to minimize the release of anesthetic from a bottle to the environment surrounding a vaporizer. These devices, however, have failed to effectively minimize the release of anesthetic.

### BRIEF DESCRIPTION OF THE DRAWINGS

FIG. 1 illustrates an anesthetic agent container according to the invention;

FIG. 2A illustrates a container closure according to a first embodiment of the invention in a closed position and the container and vaporizer disconnected from each other;

FIG. 2B is a cross-section of the container in FIG. 2A taken along the line A-A in FIG. 2A;

FIG. 3A illustrates the container closure in an open position and the container and vaporizer connected to each other;

FIG. 3B; is a cross-section of the container in FIG. 3A taken along the line A-A in FIG. 3A;

FIG. 4A illustrates a perspective view of the membrane of the closure system in a closed position;

FIG. 4B illustrates a bottom view of the membrane of FIG. 4A;

FIG. 4C is a cross-section of the membrane of FIG. 4B taken along the line A-A in FIG. 4B;

FIG. 4D illustrates a side view of the membrane of FIG. 4A;

FIG. 4E illustrates a close-up side view of a portion of the membrane of FIG. 4D;

FIG. 4F illustrates a side view of the membrane of the closure system in an open position;

FIG. 5A illustrates a portion of a container and a container closure according to a second embodiment of the invention in a closed position;

FIG. 5B illustrates a cross-sectional view of a portion of a container and a container closure taken along the line A-A in FIG. 5A according to a second embodiment of the invention in a closed position;

FIG. 5C illustrates a close-up view of a portion of FIG. 5B;

FIG. 5D illustrates a container closure according to a second embodiment of the invention in a closed position;

FIG. 5E illustrates a container closure according to a second embodiment of the invention in an open position;

FIG. 6A illustrates a container closure according to the second embodiment of the invention in an open position and the container and vaporizer connected to each other;

FIG. 6B is a cross-section of the container in FIG. 7A taken along the line A-A in FIG. 7A;

FIG. 7A illustrates the container closure in a closed position and the container and vaporizer disconnected from each other; and

FIG. 7B; is a cross-section of the container in FIG. 7A taken along the line A-A in FIG. 7A.

### DETAILED DESCRIPTION OF THE INVENTION

The accompanying drawings are intended to provide further understanding of the invention and are incorporated in and constitute a part of the description. The drawings illustrate an embodiment.

The drawings should not be taken as implying any necessary limitation on the scope of the claimed invention. The drawings are given by way of non-limitative example to explain the nature of the embodiment.

For a more complete understanding of the embodiment, reference is now made to the following description taken in conjunction with accompanying drawings.

The various features of novelty which characterize the invention are pointed out specifically in the claims which are a part of this description. For a better understanding of the invention, reference should be made to the drawings and descriptive matter in which there are illustrated and described embodiments.

For purposes of the description hereinafter, the term “upper” and “lower” shall relate to the embodiment as it is oriented in the drawings and are only used for descriptive purposes. The invention can be oriented in any manner.

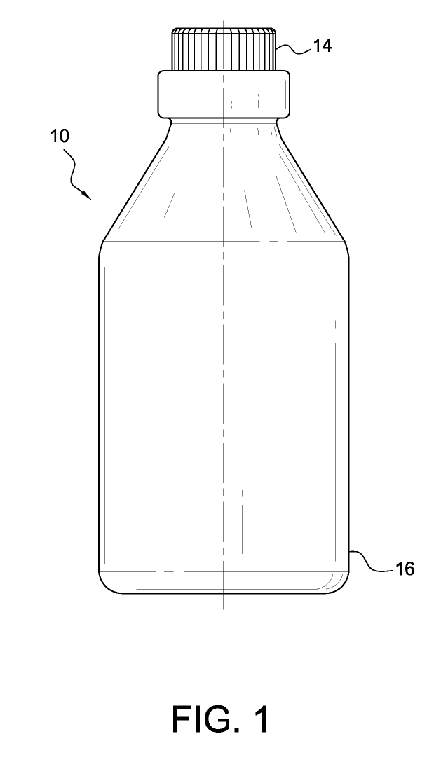

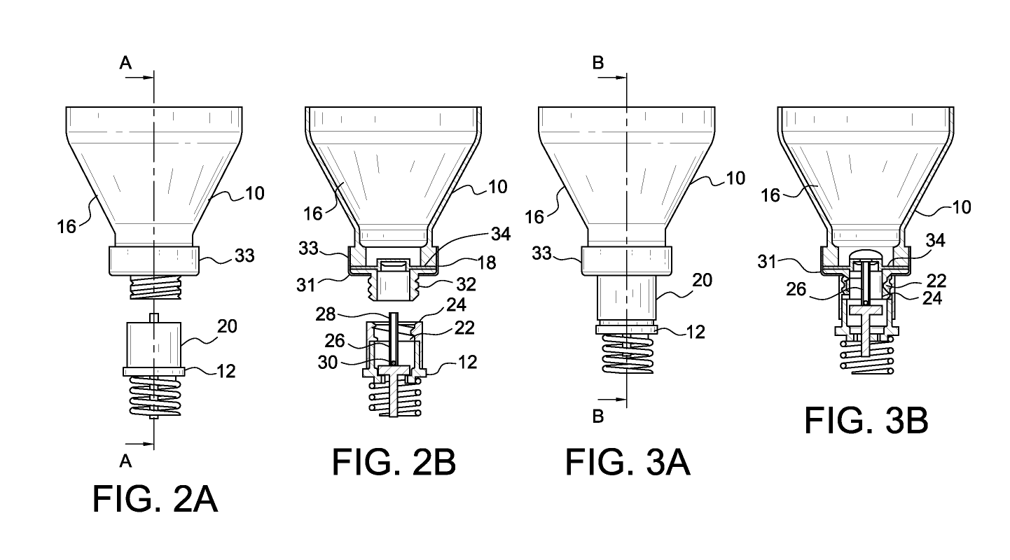

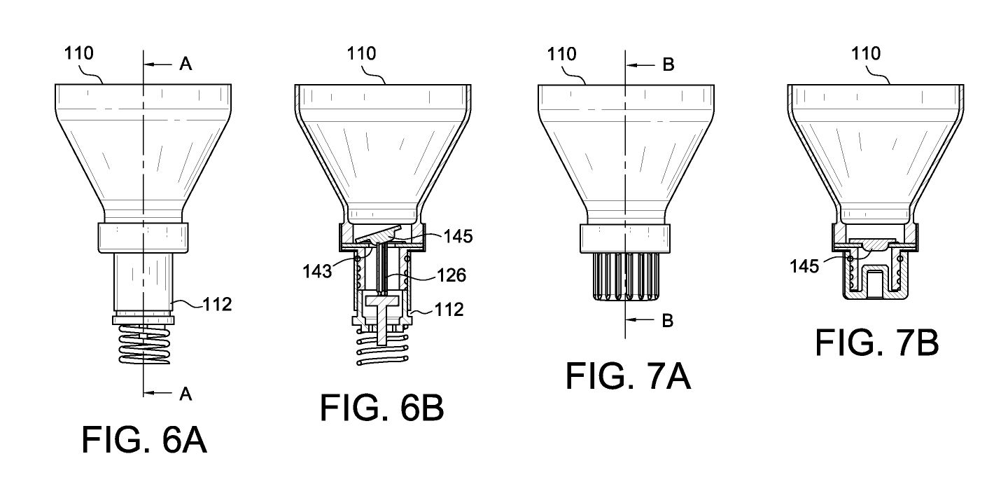

Referring now to the drawings, wherein like numerals designate identical or corresponding parts throughout the referred views, FIGS. 1-7B show various embodiments of an apparatus connecting a container 10 to a machine 12 (only a portion of which is illustrated). With reference to FIGS. 1-4F, the container 10 includes a cap 14 and a reservoir 16, such as a glass bottle capable of holding a liquid such as a volatile anesthetic. As shown in FIGS. 2A- 3B, at one end, the reservoir 16 includes an opening 18 through which anesthetic may be transferred from the reservoir 16 to the machine 12 when it is desired to transfer the anesthetic from the reservoir 16. In this embodiment, the machine is a vaporizer 12 that dispenses the anesthetic to a person undergoing a medical procedure.

The vaporizer 12 includes a nozzle 20 surrounding an opening 22 and having an interior threaded portion 24. The vaporizer 12 may be equipped with a hollow receiving member 26 such as a tube, rod, valve or needle secured within the opening 22 of the nozzle 20. The hollow receiving member 26 is adapted to carry anesthetic from a first end 28 to a second end 30 in order to supply anesthetic to the vaporizer 12.

FIGS. 2A and 2B illustrate the container 10 and vaporizer 12 disconnected from each other. FIGS. 3A and 3B illustrate the container 10 and vaporizer 12 connected to each other. Container 10 includes a base 31 at the opening 18 of reservoir 16. Base 16 includes a threaded exterior 32 for cooperating with an interior threaded surface (not shown) of cap 14. Base 16 is attached to reservoir 16 by a crimp band 33. However, any appropriate attachment mechanism can be used, such as a screw thread or adhesive attachment. When it is desired to transfer the anesthetic from the reservoir 16 to the vaporizer 12, the cap 14 is removed and the threaded exterior 32 of the base 31 of reservoir 16 is connected to the interior threaded portion 24 of the nozzle 20.

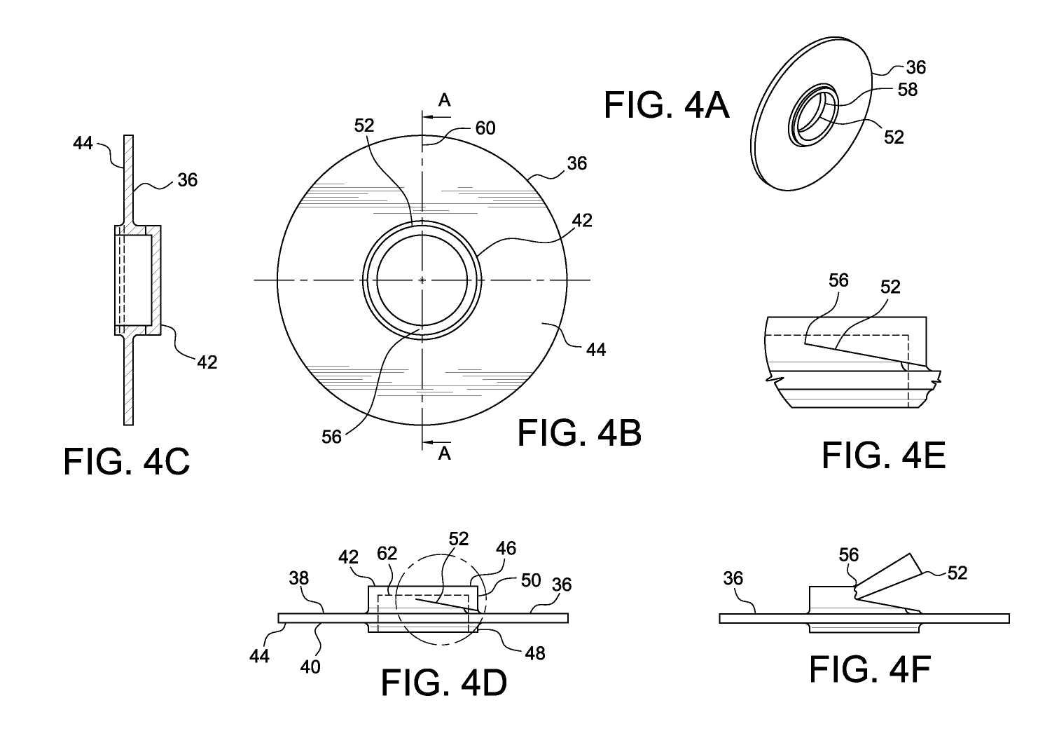

The container 10 includes a container closure member 34 for selectively allowing the volatile anesthetic to leave the reservoir 16. Base 16, crimp band 33 and closure member 34 define a closure device for reservoir 16. The container closure member 34 includes a membrane 36 (FIGS. 4A-4F). The membrane 36 may be made from a plastic material or a rubbery material such as FDA grade ethylene propylene diene M-class “EPDM” rubber. Membrane 36 has an upper surface 38, a lower surface 40, a cylindrical center portion 42 surrounded by a flanged outer portion 44. Cylindrical center portion 42 includes a closed end 46, open end 48 and a cylindrical side wall 50 therebetween. Closed end 46 is disposed in a plane above the upper surface 38. Open end 48 extends below lower surface 40. Membrane 36 is disposed across the opening 18 of reservoir 16 between base 31 and opening 18. Membrane 36 further includes a slit 52 through a portion of cylindrical side wall 50. Because of the cylindrical shape of side wall 50, slit 52 is substantially curved having a first end 54, a second end 56 and a curved central portion 58 therebetween. Curved central portion 58 extends greater than 180° around side wall 50 such that first end 54 and second end 56 are disposed past a midline through 60 cylindrical center portion 42. Curved central portion 58 is angled downwards from first end 54 and second end 56 to the upper surface 38 of membrane 36. When the container 10 is not connected to the vaporizer 12, pressure from within container 10 pushes on closed end 46 sealing the slit 52 in a closed position. FIGS. 2B and 4A-4E show slit 52 in a closed position.

When the threaded exterior 32 of container 10 is inserted into the interior threaded portion 24 of the nozzle 20 (FIG. 3B), the receiving member 26 engages an interior surface 62 of closed end 46 a center portion thereof causing the slit 52 to open like a hinge about first end 54 and second end 56. In this position, the anesthetic flows through the receiving member 26 from the first end 28 to the second end 30, and in so doing the anesthetic flows by gravity from the reservoir 16 to the vaporizer 12.

Once a desired amount of the anesthetic has been emptied from the reservoir 16 to the vaporizer 12, the container 10 may be removed from the vaporizer 12. As the container 10 is removed, the receiving member 26 disengages interior surface 62 of closed end 46. When the receiving member 26 is no longer extended with the interior surface 62, the back pressure of container 10 closes the slit 52. In this manner, any anesthetic remaining in the reservoir 16 is prevented from escaping from the reservoir 16.

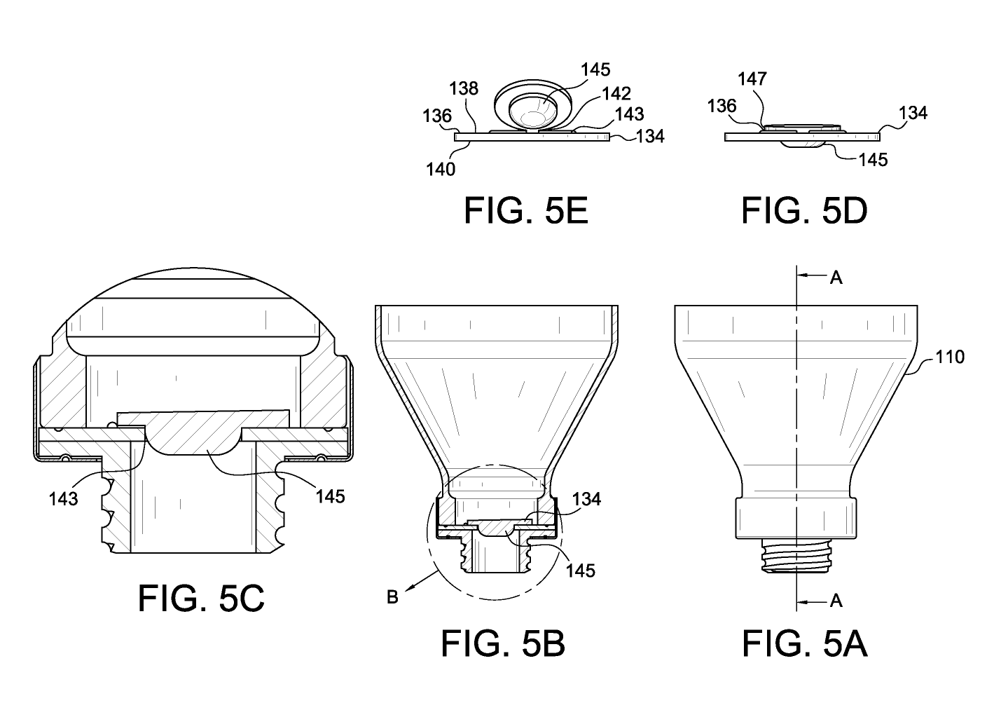

FIGS. 5A to 7B illustrate an embodiment having a container 110, a vaporizer 112, a receiving member 126 and a container closure member 134. As illustrated in FIGS. 5D and 5E, container closure member 134 includes a membrane 136 having an upper surface 138, a lower surface 140, a hinged closure portion 142 and a central circular opening 143 surrounded by a flanged outer portion 144. Hinged closure portion 142 includes a central dome portion 145 surrounded by a flanged portion 147. Flanged portion 147 is hingedly attached at one end 149 to flanged outer portion 144 of membrane 136 such that when closed central dome portion 145 is inserted into central circular opening 143 and a lower surface of flanged portion 147 seals with an upper surface 138 of membrane 136.

When the container 110 connected to vaporizer 112, the receiving member 126 engages a lower surface 149 of central dome portion 145 thereby causing the central dome portion 145 to disengage from the central circular opening 143. In this position, the anesthetic flows through the receiving member 126 by gravity from the container 110 to the vaporizer 112.

Once a desired amount of the anesthetic has been emptied from the container 110 to the vaporizer 112, the container 110 may be removed from the vaporizer 112. As the container 110 is removed, the receiving member 126 disengages from central dome portion 145 and the central dome portion 145 engages within the central circular opening 143.

It is to be understood that the descriptions of the invention have been simplified to illustrate characteristics that are relevant for a clear understanding of the invention. Those of ordinary skill in the art may recognize that other elements or steps are desirable or required in implementing the invention. However, because such elements or steps are well known in the art, and because they do not facilitate a better understanding of the invention, a discussion of such elements or steps is not provided herein. The disclosure herein is directed to all such variations and modifications to such elements and methods known to those skilled in the art.

It is to be understood that the invention may assume various alternative orientations and step sequences, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. An anesthetic container for transferring anesthesia to a vaporizer, the container comprising:
a reservoir adapted for containing the liquid anesthesia,
an opening at one end of the reservoir; and
a hinged closure member attached to the reservoir, the hinged closure member being moveable between an open position and a closed position, wherein the hinged closure member is adapted to be moved from the closed position to the open position by a receiving member of a vaporizer when the anesthetic container is attached to the vaporizer for transfer of liquid anesthetic from the anesthetic container to the vaporizer, and wherein the hinged closure member is moved to the closed position upon withdrawal of the receiving member.

2. The container of claim 1, wherein the hinged closure member includes a membrane having an upper surface, a lower surface, and a cylindrical center portion surrounded by a flanged outer portion.

3. The container of claim 2, wherein the cylindrical center portion includes a closed end, an open end and a cylindrical side wall therebetween

4. The container of claim 3, wherein the cylindrical center portion further includes a slit through a portion of cylindrical side wall.

5. The container of claim 1, wherein the hinged closure member includes a membrane having an upper surface, a lower surface, and a central opening and further includes a hinged closure portion having a central dome portion surrounded by a flanged outer portion.

6. The container of claim 5, wherein the flanged portion is hingedly attached at one end to the flanged outer portion of the membrane.

7. The container of claim 6, wherein the flanged portion the central dome portion is inserted into central circular opening when in the closed position.

8. The container of claim 1, further comprising a connection mechanism for connecting the container to a vaporizer.

9. The container of claim 8, wherein the connection mechanism comprises a base attached to the reservoir and adapted for connection to a vaporizer.

10. The container of claim 9, wherein the base has threads formed on an exterior surface adapted for connection to a threaded opening in the vaporizer.

11. The container of claim 10, wherein the container further includes a cap connectable to the threads when the container is not connected to a vaporizer.

12. A closure device for coupling an anesthetic container to a vaporizer, the device comprising:
a base adapted for connection to an opening of an anesthetic container; and
a hinged closure member attached to the reservoir, the hinged closure member being moveable between an open position and a closed position, wherein the hinged closure member is adapted to be moved from the closed position to the open position by a receiving member of a vaporizer when an anesthetic container is attached to a vaporizer for transfer of liquid anesthetic from the anesthetic container to the vaporizer, and wherein the hinged closure member is moved to the closed position upon withdrawal of the receiving member.

13. The device of claim 12, wherein the hinged closure member includes a membrane having an upper surface, a lower surface, and a cylindrical center portion surrounded by a flanged outer portion.

14. The device of claim 12, wherein the cylindrical center portion includes a closed end, an open end and a cylindrical side wall therebetween.

15. The device of claim 14, wherein the cylindrical center portion further includes a slit through a portion of cylindrical side wall.

16. The device of claim 12, wherein the hinged closure member includes a membrane having an upper surface, a lower surface, and a central opening and further includes a hinged closure portion having a central dome portion surrounded by a flanged outer portion.

17. The device of claim 16, wherein the flanged portion is hingedly attached at one end to the flanged outer portion of the membrane.

18. The device of claim 17, wherein the flanged portion the central dome portion is inserted into central circular opening when in the closed position.

19. The device of claim 12, further comprising a connection mechanism for connecting a container to a vaporizer.

20. The device of claim 19, wherein the connection mechanism comprises threads formed on an exterior surface of the base and adapted for connection to a threaded opening in the vaporizer.

21. The device of claim 20, further comprising a cap connectable to the threads when the container is not connected to a vaporizer.
